(19) United States
(12) Patent Application Publication
(10) Pub. No.: US 2009/0317445 A1
(43) Pub. Date: Dec. 24, 2009

(54) DRUG DELIVERY SYSTEM WITH THERMOSWITCHABLE MEMBRANES

(75) Inventors: Michel Paul Barbara Van Bruggen, Eindhoven (NL); Hendrika Cecilia Krijnsen, Eindhoven (NL); Ventzeslav Petrov Iordanov, Eindhoven (NL); Anna-Maria Janner, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,036

(22) PCT Filed: Jul. 13, 2007
(86) PCT No.: PCT/IB2007/052809

§ 371 Date: Jan. 26, 2009

(30) Foreign Application Priority Data
Jul. 27, 2006 (EP) 06117939.6

(51) Int. Cl. A61K 9/00

(52) U.S. Cl. 424/423

(57) ABSTRACT

The present invention provides a device for controlled release of molecules. The device is particularly suitable for controlled release of therapeutic drugs to a patient. The device includes a housing with an opening for release of the molecules from the housing. The housing also comprises a reservoir for containing the molecules, in particular therapeutic drugs. The reservoir is arranged in the housing to allow release of the molecules through the opening. The device also comprises at least one thermoswitchable membrane and at least one heating element for at least partially heating the membrane. The device is configured for modulating the release of the molecules at the opening by heating the membrane, using the heating element. Optionally, the device further comprises a pressure element for providing pressurized release of the molecules from the device. In this way, the drug can be delivered to a patient in a pulsatile fashion. The present invention also provides a method for modulating the release of molecules, using such a device.

The present invention relates to a device for controlled release of molecules. In particular, the present invention relates to a device for delivering one or more drugs to, in particular, a human or animal body. The device may be applied transdermally or may be implanted in the human or animal body.

Drug delivery systems have thus far had a great impact on medical technology. The efficacy of drug treatment is often dependent upon the mode of drug delivery. Localized drug delivery is oftentimes preferred, since it traverses limitations associated with systemic drug delivery. Such limitations include rapid drug inactivation and/or ineffectual drug concentrations at the site of treatment. Moreover, systemic drug delivery may lead to undesired cytotoxic effects at tissue regions other than that to be treated.

Implantable drug delivery systems greatly improve the performance of many existing drugs and enable the use of entirely new therapies. They allow for localized delivery of drugs and therefore prevent many side effects of drug therapies. Moreover, implantable drug delivery systems allow for administration of otherwise insoluble, unstable or unavailable therapeutic compounds to a patient, a reduction of the amount of such compounds to be administered and improvement of compliance for a patient receiving drug therapy by reducing the chances of missing or erring in a dose.

Presently, many small-scale systems are available for in vivo drug delivery. They are e.g. reviewed in LaVan D. et al. (LaVan D. A., McGuire T., Langer R. 2003. Small-scale systems for in vivo drug delivery. Nature Biotechnology, vol. 21, no. 10, pp. 1184-1191). They include microfabricated devices, diffusion chambers, nanoparticles, and ‘smart’ devices.

US 2002/0187260 describes a microchip device for the controlled release or exposure of molecules. The device contains reservoirs, which are capped by a reservoir cap. The reservoir cap includes a membrane, a reservoir cap, a plug, or any other physical or chemical structure suitable for separating the contents of a reservoir from the environment outside the reservoir. The reservoir cap is selectively removed or permeabilized, preferably selectively disintegrated. In passive devices, the reservoir cap is formed from a material that degrades, dissolves, or disintegrates over time. In active devices, the reservoir cap includes any material that can be disintegrated or permeabilized in response to an applied stimulus. In a preferred embodiment of the device, the reservoir cap is a thin metal, e.g. gold, silver, copper or zinc, membrane that disintegrates by exposure to an electrochemical reaction started by the application of an electric potential. The disintegration is irreversible.

US 2004/0032187 discloses a device for controlled release of drugs. The device consists of a body having a reservoir for containing the drug molecules. The reservoir is formed with a barrier impermeable to the molecules, thereby preventing their release. An acoustic transducer for converting an acoustic signal received by it into an electrical signal, is attached to the body. The electrical signal leads to barrier permeabilization, and therefore release of the molecules from the reservoir. In an embodiment, an electrical potential converts the molecules stored within the reservoir into an active, barrier-permeable form of molecules. In another embodiment, the electrical potential generated by electrodes causes partial or full disintegration of the barrier. In the latter case, the barrier can be composed of conductive materials that are capable of dissolving into solution or forming soluble compounds or ions upon the application of an electrical potential. Such materials include metals such as copper, gold, silver and zinc and some polymers. The disintegration is irreversible.

Polymers exist that exhibit a critical solution temperature (cst). The critical solution temperature is the temperature at which the gel displays a phase transition from an extended and soluble conformation to a globular collapsed and insoluble conformation. These polymers belong to the class of thermoswitchable polymers. Polymers that display this behaviour upon an increase of the temperature exhibit a lower critical solution temperature (lcst), and polymers that display this behaviour upon a decrease of the temperature exhibit an upper critical solution temperature (ucst). Both lcst and ucst can be tailored by chemical modifications of the polymer systems.

Thermoswitchable polymer systems are presently used for drug delivery purposes, in particular in so-called drug depot formulations. In the extended conformation, the polymer chains are fully solvated, leaving an open and permeable structure, whereas in the collapsed state the polymer structure becomes relatively impermeable. A drug depot formulation consists of various compounds, but the minimum formulation requirements include a solvent, optionally a co-solvent, a drug (or cocktail of drugs) and the dissolved polymer or a precursor of the polymer. The formulation is injected (often cooled) into the body. Inside the body the formulation starts gelling as the lower critical solution temperature is passed. In the gelled form, the drug(s) can only slowly diffuse out of the matrix, giving a sustained drug release over a prolonged period of time. However, this drug delivery system does not allow for a pulsatile delivery profile. In addition, the only way to stop the delivery of the drug is by removal of the gel (implant) from the body.

It is an object of the present invention to provide a device for the controlled release of molecules that allows for a pulsatile delivery profile of the molecules. The invention in particular makes use of a thermoswitchable polymer membrane, the permeability of which can be reversibly modulated by increasing or decreasing the temperature of the polymer, using a heating element that is located within the device.

In an aspect, the present invention provides a device for controlled release of molecules. The device is particularly suitable for controlled release of therapeutic drugs to a patient. The device includes a housing with an opening for release of the molecules from the housing. The housing also comprises a reservoir for containing the molecules, in particular therapeutic drugs. The reservoir is arranged in the housing to allow release of the molecules through the opening. The device also comprises at least one thermoswitchable membrane and at least one heating element for at least partially heating the membrane. The device is configured for modulating the release of the molecules at the opening by heating the membrane, using the heating element.

Whereas in the prior art the release of the molecules can be controlled only in that such a release can be switched on, the device according to the present invention allows a pulsatile release of the molecules, in particular by making use of the thermoswitchable response of a polymer to temperature.

In a further aspect, the present invention provides a method for modulating the release of molecules, using a device according to the present invention.

Hereinafter, the present invention and further advantageous features are described and elucidated in more detail with reference to the appended drawings showing non-limiting embodiments of the invention, wherein

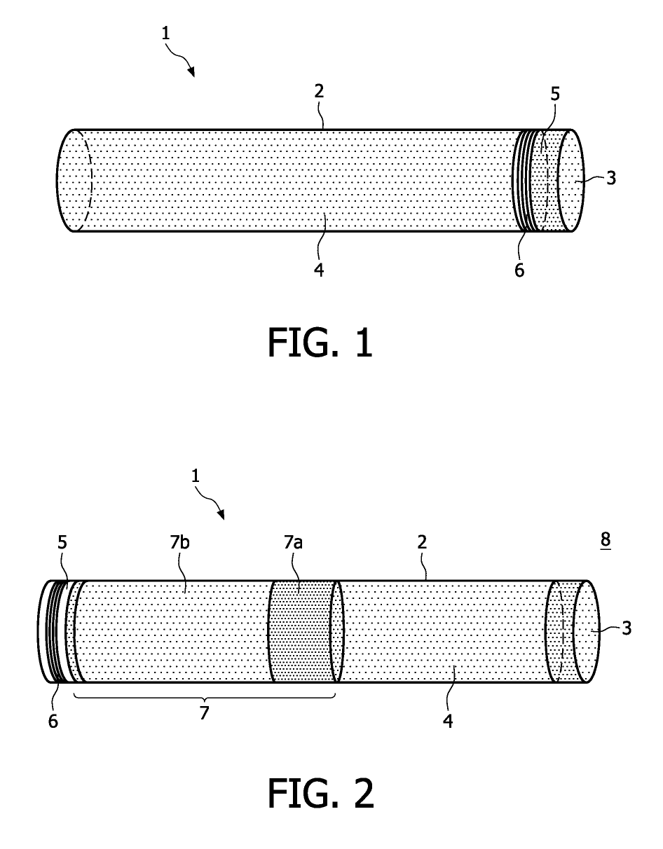

FIG. 1 schematically shows a side view of a first embodiment of a device for controlled release of molecules according to the present invention;

FIG. 2 schematically shows a side view of a second embodiment of a device for controlled release of molecules according to the present invention;

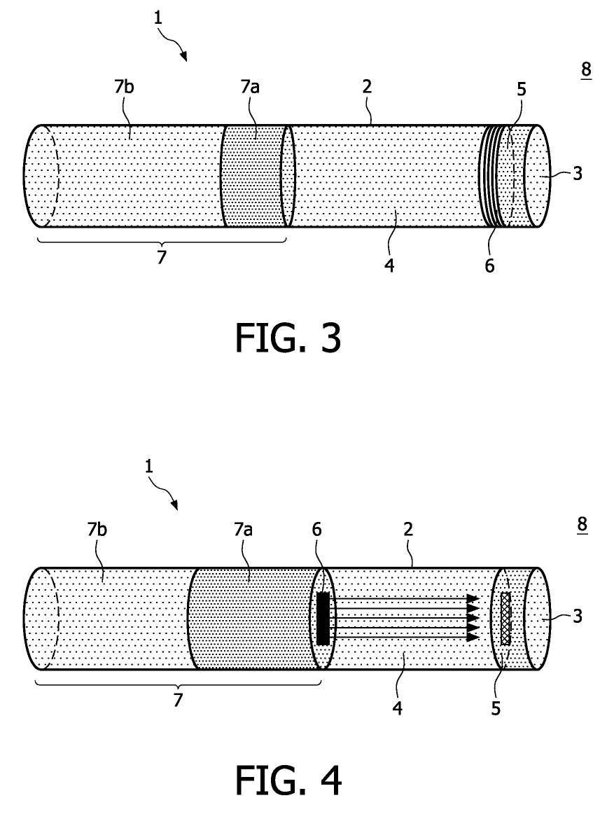

FIG. 3 schematically shows a side view of a third embodiment of a device for controlled release of molecules according to the present invention;

FIG. 4 schematically shows a side view of a fourth embodiment of a device for controlled release of molecules according to the present invention.

The present invention relates to a device for controlled release of molecules, including a housing having an opening, said housing comprising at least one reservoir for containing the molecules, the reservoir being arranged in the housing to allow release of the molecules through the opening, said device further comprising at least one thermoswitchable membrane, and at least one heating element for at least partially heating said membrane, the device being configured for modulating the release of the molecules at the opening by heating the membrane, using the heating element.

The housing preferably is fabricated from a material that is impermeable to the molecules to be released and to the surrounding fluids of the device, for example, water, blood, electrolytes or other solutions. Examples of suitable materials include ceramics, e.g. Al2O3, metals such as titanium and stainless steel, and polymers. It is preferred that the housing is made form a biocompatible material.

The molecules may be any molecules that require release to an environment. They may be therapeutic drugs, hormones, enzymes, antibodies and the like.

The device also comprises at least one thermoswitchable membrane. As used herein, the term “thermoswitchable membrane” or “membrane” refers to a membrane that is reversibly, more or less permeable as the temperature of its constituent increases or decreases.

The device further comprises at least one heating element for at least partially heating the membrane. Heating of the membrane by the heating element will increase or decrease its permeability, allowing for release of the molecules through the membrane, or ending release of the molecules through the membrane, respectively. Non-limiting examples of suitable heating elements include photon-emitting elements such as a LED and a laser diode, an electrical resistance heating element, an ultrasonic transducer, and an electromagnetic coil. In case the heating element is a photon-emitting element, the membrane may optionally comprise photon-sensitive particles. In case the heating element is an electromagnetic coil, the membrane may comprise magnetic material.

The device is configured for modulating the release of the molecules at the opening by heating the membrane, using the heating element.

In an embodiment of the device according to the present invention, the reservoir for containing the molecules is at least partially formed by the thermoswitchable membrane, and the thermoswitchable membrane is arranged at the opening to allow release of the molecules through the membrane and the opening. Heating of the membrane by the heating element increases or decreases its permeability, thereby allowing modulation of the release of the molecules from the reservoir into the environment of the device.

In a preferred embodiment, the housing further comprises a pressure element, the pressure element generating a release pressure and the pressure element being arranged in the housing to allow pressurized release of the molecules through the opening.

The pressure element may be any pressure element known in the art. Such pressure elements are well known to a person skilled in the art. For example, the pressure element may be a system composed of a pressurizing compartment, and a piston or any other barrier that can move within the housing. Non-limiting examples of such pressure elements are a so-called pressure engine and piston, a system composed of a so-called osmotic engine and a piston, a spring with a movable barrier, and the like. The pressure element is preferably arranged in the housing to allow movement of the barrier between the pressurizing compartment and the reservoir. It is preferred that when the pressure in the pressurizing compartment increases, the barrier moves to decrease the volume of the reservoir, and molecules are released under pressure from the device.

In another embodiment of the device according to the present invention, the housing further comprises a pressure element, the pressure element generating a release pressure and the pressure element being arranged in the housing to allow pressurized release of the molecules through the opening, the pressure element being at least partially formed by the membrane, the membrane being in contact with an environment.

As discussed above, an example of such a pressure element is an osmotic pressure element. Such osmotic pressure element (or osmotic engine) could e.g. be formed by a pressurizing compartment, the pressurizing compartment being arranged in the housing, the housing preferably having two openings: one opening for allowing release of the molecules, and one opening for allowing modulation of the pressurizing compartment. The pressurizing compartment is preferably separated from the reservoir in the housing by means of a movable barrier. An example of such a barrier is a piston. Modulation of the pressurizing compartment advantageously takes place by an influx of solution, preferably water, from the environment into the pressurizing compartment when the membrane is permeabilized. Therefore, the pressurizing compartment is at least partially formed by the thermoswitchable membrane, the membrane being configured in such a way as to allow the influx of water from the environment upon permeabilization of the membrane. Upon increasing the permeability of the membrane, an influx of water takes place into the pressurizing compartment, causing a movement of the barrier into the direction of the reservoir. This causes release of the molecules from the reservoir via the opening in the housing and an outlet, the outlet for example being formed by a mechanical valve opening when pressurized or a porous membrane, flow restrictor, and the like.

The environment may be any environment, but is preferably a human or animal body, more preferably a human body. In the case of transdermal drug delivery, the environment is preferably a skin, more specifically an epidermal layer.

In an embodiment, the membrane comprises a thermoswitchable polymer. Thermoswitchable polymers typically exhibit a critical solution temperature (cst). The critical solution temperature is the temperature at which the gel displays a phase transition from an extended and soluble conformation to a globular collapsed and insoluble conformation. Polymers that display this behaviour upon an increase of the temperature exhibit a lower critical solution temperature (lcst), and polymers that display this behaviour upon a decrease of the temperature exhibit an upper critical solution temperature (ucst). Both lcst and ucst can be tailored by chemical modifications of the polymer systems. The change of the swelling ratio (defined as the absorbed mass of water divided by the dry mass of polymer) of the polymer upon passing the cst can be chemically tailored, e.g. by changing the crosslink density of the polymer network. In the extended conformation, the polymer chains are fully solvated, leaving an open and permeable structure, whereas in the collapsed state the polymer structure becomes relatively impermeable. Thermoswitchable polymers include poly-N-isopropylamide (PNIPAAm) and copolymers thereof, polyoxyethylene trimethylol-propane distearate and poly-∈-caprolactone. The critical solution temperature may be determined by measuring the polymer volume as a function of temperature.

A release of molecules that increases upon an increase of the temperature is referred to as positive controlled release (pcr) and is attained when the polymer exhibits an upper critical solution temperature (ucst). The opposite, i.e. a decrease of the release at increasing temperature, is referred to as a negative controlled release (ncr) and is attained when the polymer exhibits a lower critical solution temperature (lcst). For example, pure PNIPAAm having a lcst exhibits ncr, whereas a copolymer of NIPAAm and acrylamide exhibits pcr.

The transition from a collapsed state to a swollen state takes place over a certain temperature range ΔT. Within this range, each temperature corresponds to a certain swollen state of the gel. As a consequence, the permeability will gradually change within the range ΔT. In this way, the drug release rate can be tuned.

Thus, in one embodiment, the thermoswitchable polymer is a polymer having an upper critical solution temperature. Upon heating of a ucst polymer membrane, the membrane displays a phase transition from a globular collapsed and insoluble conformation to an extended and soluble conformation. In its globular collapsed and insoluble conformation, the polymer is impermeable to the molecules contained within the reservoir, whereas in its extended and soluble conformation, the molecules may pass through the membrane to be released to the environment of the device. When such ucst polymer membrane is employed in the device according to the present invention, the membrane is essentially impermeable to the molecules contained within the reservoir when the membrane is not heated. Upon heating, the membrane becomes permeable to the molecules, and the molecules may be released to the environment. The use of a ucst polymer is particularly suitable when occasional (pulsatile) administration of molecules is desired. It allows for a normally closed valve that can be temporarily opened.

In another embodiment, the thermoswitchable polymer is a polymer having a lower critical solution temperature. Upon heating of an lcst polymer membrane, the membrane displays a phase transition from an extended and soluble conformation to a globular collapsed and insoluble conformation. When such an lcst polymer membrane is employed in the device according to the present invention, the membrane is essentially permeable to molecules contained within the reservoir when the membrane is not heated. Thus, the molecules are released to the environment. Upon heating, the membrane becomes impermeable to the molecules, and release of the molecules stops. The use of an lcst polymer is particularly suitable for frequent and prolonged administration, as the system behaves like a normally open valve that can be temporarily closed.

In an embodiment, the thermoswitchable polymer is selected from poly-N-isopropylamide and copolymers thereof, polyoxyethylene trimethylol-propane distearate, and poly-∈-caprolactone.

In an embodiment, the heating element is a photon-emitting element. In such a case, the membrane is photon-sensitive, e.g. by comprising photon-sensitive particles, dyes, or by having an absorption maximum at the wavelength of the light source. Non-limiting examples of a photon-emitting element include an LED, laser diode, and the like.

In an embodiment, the heating element (photon-emitting element) is selected from a LED source and a laser diode.

In a further embodiment, the membrane comprises photon-sensitive particles. It is known in the art that thermoswitchable polymer hydrogels can contain light-absorbing particles (herein also referred to as ‘photon-sensitive particles’) that are equally distributed and fixed into the polymer structure. The thermo-switchable polymers can be switched by light when the wavelength of the light is in the region in which the particles absorb, leading to a decrease of light intensity and a rise of the local temperature. The advantage of such an approach is that the molecules within the reservoir are not in direct contact with a heating element, which may lead to drug stability problems of the formulation over time. Such photon-sensitive particles typically consist of an inner core with diameter d and dielectric constant ∈₁ and an outer shell with thickness t and dielectric constant ∈₂. The inner core can be silica, the outer core gold. FIG. 8 gives the extinction profiles for various values of t. The diameter d of the inner core can e.g. be 50 to 150 nm, and the thickness of the outer shell may vary between 2 and 30 nm, preferably between 3 and 25 nm, more preferably between 3 and 30 nm. In case the thermoswitchable membrane is a membrane prepared from a thermoswitchable polymer, the light-absorbing particles may e.g. be dispersed in such a polymer. The average (LED) power supplied to the thermoswitchable membrane may typically be varied via the pulse frequency ω and pulse duration τ.

In another embodiment, the heating element is an electrical resistance heating element. Such an electrical resistance heating element is preferably at least partially arranged in contact with the membrane. The average current supplied to the thermoswitchable membrane may typically be varied via the pulse frequency ω and pulse duration τ.

The molecule release rate can be tuned by varying the pulse frequency ω and pulse duration τ independently.

The device may further comprise a control element for controlling the heating element. Such a device may be any device known in the art, but is preferably a microprocessor. The microprocessor may optionally be controlled from outside the device, e.g. using a remote control.

In a further embodiment, the housing comprises a plurality of reservoirs, each reservoir being at least partially formed by the respective membrane, each reservoir containing molecules of a specific type and being capable of releasing the molecules upon heating of the heating element. The reservoirs may jointly comprise one membrane, or each reservoir may comprise its own membrane. In the latter case, the membranes may be of the same or a different composition. The device may further comprise one heating element heating all the membranes of the respective reservoirs simultaneously, or may comprise one heating element that can be specifically directed to the membrane to be heated.

In an embodiment, the respective membrane of each reservoir is at least partially heatable independently by a respective heating element. In this way, each reservoir can be handled separately, and multiple types of molecules can be released independently of one another.

In a further aspect, the present invention relates to a method for modulating the release of molecules from a reservoir, using a device according to the invention.

In an embodiment, the molecules are released onto or in a human or animal body. Thus, the device can be used to deliver drugs to a patient in need thereof.

In an embodiment, the device is implanted into a human or animal body. In such a case, the human or animal body forms the environment of the device.

In another embodiment, the device is applied transdermally, the opening being in contact with an epidermis. The epidermis forms the top layer of the skin. In such an embodiment, the molecules are to traverse the skin of the human or animal in order to be taken up by the human or animal body.

The invention will hereinafter be described in more detail with reference to the accompanying Figures. In the Figures, like reference numerals refer to like components.

Referring now to the drawings, FIG. 1 illustrates a side view of a first embodiment of a device (1) for controlled release of molecules according to the present invention. The device (1) includes a housing (2), which housing (2) has an opening (3) that allows for release of the molecules from the device (1). The device (1) contains a reservoir (4) for containing the molecules that are to be released from the device (1). Thus, the reservoir (4) is arranged in the housing (2) to allow release of the molecules through the opening (3). The reservoir (4) is at least partially formed by a thermoswitchable membrane (5). The membrane (5) is arranged at the opening (3) to allow release of the molecules through the membrane (5) and the opening (3).

The device (1) further contains a heating element (6) for at least partially heating the membrane (5). The heating element (6) in the embodiment of FIG. 1 is an electrical resistance heating element (hereinafter also referred to as ‘electrical resistance heating element (6)’). The electrical resistance heating element is at least partially arranged in contact with the membrane (5). This configuration allows heating of the membrane (5) by electrical resistance heating element (6). As explained above, heating of the membrane (5) by heating element (6) modulates the release of molecules. An increase in permeability of the membrane (5) will result in a release of molecules, whereas a decrease in permeability of the membrane (5) will restrict the release of molecules. Depending on the type of membrane (5) used, either result can be achieved.

In another embodiment, the membrane (5) may be activated by a photon-emitting element, e.g. a LED source or a laser diode, which may be configured in a similar fashion as shown in FIG. 4.

The diffusion rate of the molecules and the permeability of the thermoswitchable membrane determine the release rate of the molecules in this embodiment of a device according to the present invention.

FIG. 2 illustrates a side view of a second embodiment of a device (1) for controlled release of molecules according to the present invention. The device (1) includes a housing (2), which housing (2) has an opening (3) that allows for release of the molecules from the device (1). The device (1) contains a reservoir (4) for containing the molecules that are to be released from the device (1). Thus, the reservoir (4) is arranged in the housing (2) to allow release of the molecules through the opening (3). The device (1) further contains a thermoswitchable membrane (5), and a heating element (6) for at least partially heating the membrane (5). The device (1) is configured for modulating the release of the molecules at the opening (3) by heating the membrane (5), using the heating element (6). The housing (2) further comprises a pressure element (7). The pressure element (7) generates a release pressure, and the pressure element (7) is arranged in the housing (2) to allow pressurized release of the molecules through the opening (3). The pressure element (7) is at least partially formed by the membrane (5). The membrane (5) is in contact with an environment (8).

The reservoir (4) may be closed off at the opening (3) by a porous membrane, a mechanical valve, a flow restrictor or the like.

The heating element (6) in the embodiment of FIG. 2 is an electrical resistance heating element (hereinafter also referred to as ‘electrical resistance heating element (6)’). The electrical resistance heating element is at least partially arranged in contact with the membrane (5). This configuration allows heating of the membrane (5) by electrical resistance heating element (6). As explained above, heating of the membrane (5) by heating element (6) modulates the release of molecules. An increase in permeability of the membrane (5) will result in a release of molecules, whereas a decrease in permeability of the membrane (5) will restrict the release of molecules. Depending on the type of membrane (5) used, either result can be achieved.

The pressure element (7) of the second embodiment according to the present invention consists of a piston (7a) and a pressurizing compartment (7b) which is an osmotic engine. Upon increasing the permeability of the membrane (5) by heating the membrane (5) using the electrical resistance heating element (6), an influx of water into the pressurizing compartment (7b) will generate a pressure within pressurizing compartment (7b) that will result in movement of piston (7a) in the direction of the reservoir (4). Due to this pressure generated, molecules will be released from reservoir (4) through opening (3).

The device of FIG. 2 in particular consists of a single reservoir (4) that is closed off at one end by a piston (7a) and at the other end by a (non-switchable) membrane or outlet. The pressure element (7) is further formed by an osmotic engine (7b) separated from the environment by a thermoswitchable membrane (5) consisting of a thermoswitchable polymer that may be deposited onto a porous membrane or support to enhance its mechanical integrity. An example of parameters suitable for the device of FIG. 2 is the following: Area/thickness of the thermoswitchable polymer is 4 mm2/0.1 mm, density ˜1 g/ml, heat cap. ˜4.2 J/K g, max. T rise ˜12 K, power source: coin battery, 3V @ 1 mA, max. response time is (vol. polymer×density×max T raise×heat cap.)/electrical energy output=20 mJ/3 mW is about 6 sec.

In a preferred embodiment, the membrane becomes permeable to water molecules when being heated, and the osmotic pressure engine (7b) (the pressurizing compartment (7b) that is part of pressure element (7)) starts pushing the piston (7a) (the barrier that is part of pressure element (7)) in the direction of the reservoir, thereby pressurizing the reservoir (4). This may lead to release of molecules from the reservoir (4) through the opening (3).

Osmotic pressure as well as the permeability change of the thermoswitchable polymer determines the change of the release rate of the molecules, e.g. drug administration rate.

In another embodiment, the membrane (5) may be activated by a photon-emitting element, e.g. a LED source or a laser diode, which may be configured in a similar fashion as shown in FIG. 4.

Now, referring to FIG. 3, the device (1) for controlled release of molecules includes a housing (2) having an opening (3), said housing (2) comprising at least one reservoir (4) for containing the molecules, the reservoir (4) being arranged in the housing (2) to allow release of the molecules through the opening (3), said device (1) further comprising at least one thermoswitchable membrane (5), and at least one heating element (6) for at least partially heating said membrane (5), the device (1) being configured for modulating the release of the molecules at the opening (3) by heating the membrane (5), using the heating element (6). The housing (2) further comprises a pressure element (7), the pressure element (7) generating a release pressure and the pressure element (7) being arranged in the housing (2) to allow pressurized release of the molecules through the opening (3).

The heating element (6) in the embodiment of FIG. 3 is an electrical resistance heating element (hereinafter also referred to as ‘electrical resistance heating element (6)’). The electrical resistance heating element is at least partially arranged in contact with the membrane (5). This configuration allows heating of the membrane (5) by electrical resistance heating element (6). As explained above, heating of the membrane (5) by heating element (6) modulates the release of molecules. An increase in permeability of the membrane (5) will result in a release of molecules, whereas a decrease in permeability of the membrane (5) will restrict the release of molecules. Depending on the type of membrane (5) used, either result can be achieved. In another embodiment, the membrane (5) may be activated by a photon-emitting element, e.g. a LED source or a laser diode, which may be configured in a similar fashion as shown in FIG. 4.

The pressure element (7) in the embodiment of FIG. 3 is formed by a piston (7a) and a pressurizing compartment (7b) that is a pressure engine, the piston (7a) being located in between the pressure engine (7b) and the reservoir (4). However, the pressure element (7) may be any pressure element, such as those described above.

The device according to the embodiment of FIG. 3 consists of a single reservoir (4) that is closed off by a piston (7a), on the side opposite to the opening (3), and a thermoswitchable membrane (5) on the side of the opening (3). An example of parameters suitable for the device of FIG. 3 is the following: Area/thickness of the thermoswitchable polymer is 4 mm2/0.1 mm, density ˜1 g/ml, heat cap. ˜4.2 J/K g, max. T rise ˜12 K, power source: coin battery, 3V @ 1 mA, max. response time is (vol. polymer×density×max T raise×heat cap.)/electrical energy output=20 mJ/3 mW is about 6 sec.

In a preferred embodiment, the pressure engine (7b) pushes the piston (7a) in the direction of the reservoir (4) when the thermoswitchable polymer membrane (5) is heated, thus pressurizing the reservoir (4). This leads to release of molecules from the reservoir (4).

Pressure-change as well as permeability-change of the thermoswitchable polymer determines the change of the release rate of the molecules, e.g. drug administration rate.

FIG. 4 illustrates a side view of a fourth embodiment of a device (1) for controlled release of molecules according to the present invention. The device (1) includes a housing (2) having an opening (3), said housing (2) comprising at least one reservoir (4) for containing the molecules, the reservoir (4) being arranged in the housing (2) to allow release of the molecules through the opening (3), said device (1) further comprising at least one thermoswitchable membrane (5), and at least one heating element (6) for at least partially heating said membrane (5), the device (1) being configured for modulating the release of the molecules at the opening (3) by heating the membrane (5), using the heating element (6). The housing (2) further comprises a pressure element (7), the pressure element (7) generating a release pressure and the pressure element (7) being arranged in the housing (2) to allow pressurized release of the molecules through the opening (3).

The pressure element (7) in the embodiment of FIG. 4 is formed by a pressurizing compartment (7b) that is a pressure engine, and a piston (7a), the piston (7a) being located in between the pressure engine (7b) and the reservoir (4). However, the pressure element (7) may be any pressure element, such as those described above.

The heating element (6) in this embodiment is formed by a photon-emitting element, in particular a laser diode (hereinafter also referred to as ‘laser diode (6)’). The laser diode (6) is located in or on the piston (7a) of the pressure element (7) and is configured to emit photons onto the thermoswitchable membrane (see arrows) to heat it. However, the heating element may also be any other heating element, such as an electrical resistance heating element.

The device according to the embodiment of FIG. 4, in particular consists of a single reservoir (4) that is closed off by a piston (7a) on the side opposing the side where the opening (3) is located. On the side where the opening (3) is located, a thermoswitchable membrane (5) closes off the reservoir. Heating of the thermoswitchable membrane (5) is performed by local photonic heating using a laser diode (6). An example of parameters suitable for the device of FIG. 4 is the following: Area/thickness of the thermoswitchable polymer is 3.5×1.5 mm/0.1 mm, density ˜1 g/ml, the heat cap. ˜4.2 J/K g and the max. T rise ˜12 K, laser diode: 150 mW @ 30 mA & 5V, 3.5×1.5 mm, optical energy output: 6 mW, max. response time: (vol. Polymer×density×max T raise×heat cap.)/optical energy output=26 mJ/6 mW=4 sec., assuming 100% incoupling of photonic energy by light absorption by photon-sensitive (light-absorbing) particles present in the thermoswitchable polymer.

In a preferred embodiment, the pressure (e.g. osmotic, gas, spring) engine pushes laser diode (6) and piston (7a) in the direction of reservoir (4) when the thermoswitchable polymer membrane (5) is heated.

Pressure-change as well as permeability-change (e.g. photonic power) of the thermoswitchable polymer determines the release rate of the molecules, e.g. drug administration rate.

While the invention has been illustrated and described in detail in the drawings and the foregoing description, such illustration and description are to be considered illustrative and exemplary and not restrictive; the invention is not limited to the disclosed embodiments.

For example, it is possible to use various pressure elements (7) and various heating elements (6).

Other variations to the disclosed embodiments can be understood by those skilled in the art, from a study of the drawings, the disclosure, and the appended claims, and effected without departing from the spirit or scope of the invention (?). In the claims, the word “comprising” does not exclude other elements or steps, and the indefinite article “a” or “an” does not exclude a plurality. The mere fact that certain measures are cited in mutually different dependent claims does not indicate that a combination of these measures cannot be used to advantage. Any reference signs in the claims should not be construed as limiting the scope of the invention.

1. A device (1) for controlled release of molecules, including a housing (2) having an opening (3), said housing (2) comprising at least one reservoir (4) for containing the molecules, the reservoir (4) being arranged in the housing (2) to allow release of the molecules through the opening (3), said device (1) further comprising at least one thermoswitchable membrane (5), and at least one heating element (6) for at least partially heating said membrane (5), wherein the device (1) is configured for modulating the release of the molecules at the opening (3) by heating the membrane (5), using the heating element (6).

2. A device (1) according to claim 1, wherein the reservoir (4) is at least partially formed by the membrane (5), the membrane (5) being arranged at the opening (3) to allow release of the molecules through the membrane (5) and the opening (3).

3. A device (1) according to claim 1, wherein the housing (2) further comprises a pressure element (7), the pressure element (7) generating a release pressure and the pressure element (7) being arranged in the housing (2) to allow pressurized release of the molecules through the opening (3), the pressure element (7) being at least partially formed by the membrane (5), the membrane (5) being in contact with an environment (8).

4. A device (1) according to claim 2, wherein the housing (2) further comprises a pressure element (7), the pressure element (7) generating a release pressure and the pressure element (7) being arranged in the housing (2) to allow pressurized release of the molecules through the opening (3).

5. A device (1) according to claim 1, wherein the membrane (5) comprises a thermoswitchable polymer.

6. A device (1) according to claim 5, wherein the thermoswitchable polymer is a polymer having an upper critical solution temperature.

7. A device (1) according to claim 6, wherein the thermoswitchable polymer is a polymer having a lower critical solution temperature.

8. A device (1) according to claim 5, wherein the thermoswitchable polymer is selected from poly-N-isopropylamide and copolymers thereof, polyoxyethylene trimethylol-propane distearate, and poly-∈-caprolactone.

9. A device (1) according to claim 1, wherein the heating element (6) is a photon-emitting element.

10. A device according to claim 9, wherein the heating element (6) is selected from a LED source and a laser diode.

11. A device according to claim 9, wherein the membrane (5) comprises photon-sensitive particles.

12. A device (1) according to claim 1, wherein the heating element (6) is an electrical resistance heating element.

13. A device (1) according to claim 8, wherein the electrical resistance heating element is at least partially arranged in contact with the membrane (5).

14. A device (1) according to claim 1, wherein the device further comprises a control element for controlling the heating element (6).

15. A device (1) according to claim 14, wherein the control element is a microprocessor.

16. A device (1) according to claim 1, wherein the housing (2) comprises a plurality of reservoirs (4), each reservoir (4) being at least partially formed by the respective membrane (5), each reservoir (4) containing molecules of a specific type and being capable of releasing the molecules upon heating of the heating element (6).

17. A device (1) according to claim 16, wherein the respective membrane (5) of each reservoir (4) is at least partially heatable independently by a respective heating element (6).

18. A method for modulating the release of molecules from a reservoir (4), using a device (1) according to claim 1.

19. A method according to claim 18, wherein the molecules are released onto or within a human or animal body.

20. A method according to claim 18, wherein the device (1) is implanted into a human or animal body.

21. A method according to claim 18, wherein the device (1) is applied transdermally, the opening (3) being in contact with an epidermis.
